(19) United States
(12) Patent Application Publication
(10) Pub. No.: US 2014/0180325 A1
(43) Pub. Date: Jun. 26, 2014

(54) DILATOR

(71) Applicant: Terumo Kabushiki Kaisha, Shibuya-ku (JP)

(72) Inventors: Ryo OKAMURA, Shizuoka (JP); Toshiaki Shinohara, Shizuoka (JP)

(73) Assignee: Terumo Kabushiki Kaisha, Shibuya-ku (JP)

(21) Appl. No.: 14/167,403

(22) Filed: Jan. 29, 2014

Related U.S. Application Data
(63) Continuation of application No. PCT/JP2012/069366, filed on Jul. 30, 2012.

(30) Foreign Application Priority Data
Aug. 1, 2011 (JP) 2011-168776

(51) Int. Cl. A61M 29/00

(52) U.S. Cl. CPC A61M 29/00
USPC 606/191

(57) ABSTRACT

A dilator which can suppress rapid change in resistance and can perform smooth insertion into a hole includes a distal portion, an outer diameter of which increases towards a proximal end side in an axial direction; and a reduced cross-sectional area portion which is provided at least on a largest outer diameter portion of the distal portion, and is obtained by reducing a part of an outer periphery of a circular cross section, and a width of the reduced cross-sectional area portion in a chord direction is equal to or greater than a depth of the reduced cross-sectional area portion in a direction perpendicular to the chord direction.

### CROSS REFERENCES TO RELATED APPLICATIONS

This application claims priority as a continuation application under 35 U.S.C. §120 to International Application No. PCT/JP2012/069366 filed on Jul. 30, 2012, designating the U.S., and claims priority to Japanese Application No. 2011-168776 filed on Aug. 1, 2011, the entire content of both of which is incorporated herein by reference.

### TECHNICAL FIELD

The present disclosure relates to a dilator which is used for diametrical expansion of a hole communicating the outside with the inside of a living body.

### BACKGROUND DISCUSSION

The Seldinger method is a method of percutaneously introducing a catheter into the inside of a living body such as into a blood vessel. In the Seldinger method, a hole which communicates the outside with the inside of a living body is formed by puncturing the skin with a needle. Then, an elongated dilator, which has been inserted through the inside of a tubular introducer sheath through which a catheter can be inserted, is inserted into the hole.

The dilator protrudes from a distal end of the introducer sheath, and expands the diameter of the hole with a taper-shaped distal portion while passing through the hole. Accordingly, an operator senses resistance at the time of inserting the dilator into the hole, and when the operator strongly inserts the dilator to overcome such resistance, physical strain is imposed on the patient. Here, a proposal is made to reduce the resistance.

In a dilator disclosed in Japanese Registered Utility Model No. 3053402, for example, by providing a groove or a protrusion which extends in an axial direction on the surface of a tapered-shaped distal portion, decrease in contact resistance between a blood vessel and the dilator or between a tissue and the dilator is realized.

### SUMMARY

However, when resistance of the dilator using conventional technology was examined, great resistance was still sensed. The reason for this is considered that, when a largest outer diameter portion of a distal portion of a dilator passes through a hole which communicates the outside with the inside of a living body, rapid change in resistance generated before and after the passage occurs. That is, although a slit-shaped groove or protrusion having a greater depth than a width is provided on the surface of the distal portion of the dilator, the rapid change in the resistance generated when the largest outer diameter portion passes through the hole is not suppressed, and as a result an operator senses great resistance.

The present application discloses a dilator which can suppress rapid change in resistance and can be smoothly inserted into a hole which communicates the outside with the inside of a living body.

The inventors have found that rapid change in resistance generated when a dilator passes through a hole which communicates the outside with the inside of a living body is suppressed, by providing a reduced cross-sectional area portion having a predetermined shape with a reduced part of an outer periphery of a circular cross section, on a largest outer diameter portion of a distal portion of the dilator or a portion in which a rate of increase of an outer diameter in the distal portion of the dilator changes.

That is, there is provided a dilator including: a distal portion, an outer diameter of which increases towards a proximal end side in an axial direction; and a reduced cross-sectional area portion which is provided at least on a largest outer diameter portion of the distal portion, and is obtained by reducing a part of an outer periphery of a circular cross section, in which a width of the reduced cross-sectional area portion in a chord direction is equal to or greater than a depth of the reduced cross-sectional area portion in a direction perpendicular to the chord direction. The width of the reduced cross-sectional area portion in the chord direction indicates a length of a straight-line distance between intersection points of an outer periphery of a circular cross section and the reduced cross-sectional area portion, in the circular cross section of the largest outer diameter portion of the distal portion of the dilator. That is, the width of the reduced cross-sectional area portion in the chord direction indicates a width of the reduced cross-sectional area portion in a circumferential direction of the largest outer diameter portion of the distal portion of the dilator, and is a length of W1 in FIG. 1. In addition, the depth of the reduced cross-sectional area portion in a direction perpendicular to the chord direction of the reduced cross-sectional area portion is a difference between a length from the center of a circular cross section to an outer periphery on a circular arc which is not a reduced cross-sectional area portion, and a length of a perpendicular line from the center of the circular cross section to the reduced cross-sectional area portion, in the circular cross section of the largest outer diameter portion of the distal portion of the dilator. That is, the depth of the reduced cross-sectional area portion in a direction perpendicular to the chord direction of the reduced cross-sectional area portion indicates a length from the outer periphery of the circular cross section which is not a reduced cross-sectional area portion to a flat surface of the reduced cross-sectional area portion, when drawing a perpendicular line from the outer periphery of the circular cross section which is not a reduced cross-sectional area portion towards the center of the circular cross section, in the reduced cross-sectional area portion of the largest outer diameter portion of the distal portion of the dilator, and is a length of D1 in FIG. 3. In a case where the reduced cross-sectional area portion is formed on a recessed curved surface as shown in FIG. 19, the depth of the reduced cross-sectional area portion in a direction perpendicular to the chord direction of the reduced cross-sectional area portion is a difference between a length of a perpendicular line from a straight line connecting intersection points of an outer periphery of a circular cross section and the reduced cross-sectional area portion to the center of the circular cross section, and a length of a perpendicular line from the reduced cross-sectional area portion to the center of the circular cross section, and is a length of D3 in FIG. 19.

In addition, there is provided a dilator including: a distal portion, an outer diameter of which increases towards a proximal end side in an axial direction; and a reduced cross-sectional area portion which is provided at least on a portion in which a rate of increase of the outer diameter of the distal portion changes, and is obtained by reducing a part of an outer periphery of a circular cross section, in which a width of the reduced cross-sectional area portion in a chord direction is equal to or greater than a depth of the reduced cross-sectional area portion in a direction perpendicular to the chord direction.

According to the dilator configured as described above, rapid change in resistance generated when the largest outer diameter portion passes through the hole is suppressed by the reduced cross-sectional area portion, and as a result resistance sensed by an operator is reduced, and therefore smooth insertion of the dilator can be performed.

If a plurality of the reduced cross-sectional area portions are provided, rapid change in resistance generated when the largest outer diameter portion passes through the hole is more efficiently suppressed, and as a result resistance sensed by an operator is reduced, and therefore smooth insertion of the dilator can be performed.

If the reduced cross-sectional area portion is provided in at least one of positions facing in the radial direction of the cross section of the largest outer diameter portion, and an operator inserts the dilator obliquely with respect to the hole, a portion where the reduced cross-sectional area portion of the largest outer diameter portion is not provided, passes through one of portions in which rapid resistance change easily occurs and angles formed by the dilator and the hole opposing each other in the radial direction are an acute angle and an obtuse angle, since the reduced cross-sectional area portion can pass through the other portion thereof, rapid change in resistance generated when the largest outer diameter portion passes through the hole is easily and reliably suppressed, and accordingly the function of reducing the resistance sensed by an operator at the time of insertion is readily realized.

According to the other dilator configured as described above, rapid change in resistance generated when the portion in which the rate of increase of the outer diameter of the distal portion of the dilator changes passes through the hole, is suppressed by the reduced cross-sectional area portion, and as a result resistance sensed by an operator is reduced, and therefore smooth insertion of the dilator can be performed.

### BRIEF DESCRIPTION OF DRAWINGS

FIG. 1 is a perspective view of a dilator of a first embodiment.

FIG. 2 is a cross-sectional view taken along line II-II of FIG. 1.

FIG. 3 is a cross-sectional view taken along line III-III of FIG. 1.

FIG. 4 is a schematic configuration diagram showing separated dilator and introducer sheath.

FIG. 5 is a schematic configuration diagram showing combination of a dilator and an introducer sheath.

FIG. 6 is a graph showing a change in resistance when a diameter of a hole is expanded by a dilator of a Comparative Example which does not have a reduced cross-sectional area portion.

FIG. 7 is a graph showing a change in resistance when a diameter of a hole is expanded by a dilator created based on a first embodiment.

FIG. 8 is a cross-sectional view of a dilator created based on a conventional technology.

FIG. 9 is a cross-sectional view taken along line IX-IX of FIG. 8.

FIG. 10 is a cross-sectional view of another dilator created based on a conventional technology.

FIG. 11 is a cross-sectional view taken along line XI-XI of FIG. 10.

FIG. 12 is a graph showing a change in resistance when a diameter of a hole is expanded by a dilator created based on a conventional technology.

FIG. 13 is a graph showing a change in resistance when a diameter of a hole is expanded by the other dilator created based on a conventional technology.

FIG. 14 is a diagram showing a state in which a dilator is inserted into a formed on the skin.

FIG. 15 is a diagram showing an enlarged distal portion of a dilator of a second embodiment.

FIG. 16 is a cross-sectional view taken along line XVI-XVI of FIG. 15.

FIG. 17 is a cross-sectional view taken along line XVII-XVII of FIG. 15.

FIG. 18 is a cross-sectional view showing Modification Example of a dilator of an embodiment.

FIG. 19 is a cross-sectional view showing the other Modification Example of a dilator of an embodiment.

FIG. 20 is a cross-sectional view showing the other Modification Example of a dilator of an embodiment.

### DETAILED DESCRIPTION

Hereinafter, embodiments will be described with reference to the drawings. Dimensional ratios of the drawings are magnified and may be different from actual ratios, for convenience of description.

### First Embodiment

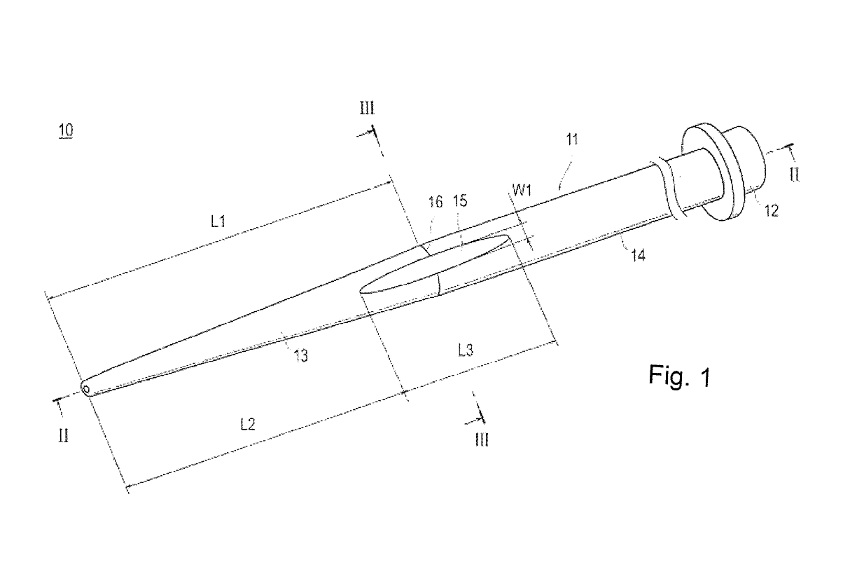

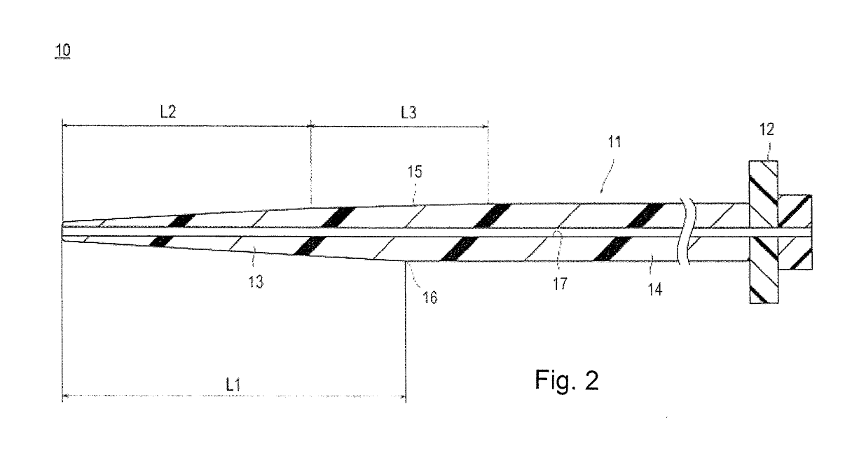

To describe FIG. 1 and FIG. 2, a dilator 10 of the embodiment is used for diametrical expansion of hole communicating the outside with the inside of a living body, and includes an elongated dilator tube 11 having flexibility, and a dilator hub 12 which is fixed to a proximal end of the dilator tube 11. In addition, the dilator 10 includes an inner cavity 17 which extends through the dilator tube 11 and the dilator hub 12 in an axial direction.

As a configuration material of the dilator tube 11, a polymer material such as a polyolefin (for example, polyethylene, polypropylene, polybutene, an ethylene-propylene copolymer, an ethylene-vinyl acetate copolymer, an ionomer, or a mixture of two or more kinds), polyolefin elastomer, a crosslinked body of polyolefin, polyvinyl chloride, polyamide, polyamide elastomer, polyester, polyester elastomer, polyurethane, polyurethane elastomer, a fluorine resin, polycarbonate, polystyrene, polyacetal, polyimide, polyetherimide, or a mixture thereof can be used, for example.

The dilator tube 11 includes a distal portion 13, an outer diameter of which increases towards a proximal end in the axial direction, and a reduced cross-sectional area portion 15 which is provided on a largest outer diameter portion 16 of the distal portion 13 and is obtained by reducing a part of an outer periphery of a circular cross section. In addition, the dilator tube 11 includes a shaft portion 14 which extends from the distal portion 13 to the proximal end side and has an approximately constant diameter along the axial direction. The outer diameter of the dilator tube 11 increases towards the proximal end side in the axial direction in the distal portion 13, and a rate of increase thereof changes in the largest outer diameter portion. In FIG. 1, the outer diameter of the shaft portion 14 is approximately constant and the rate of increase thereof is approximately zero. The reduced cross-sectional area portion 15 is provided over the portions in which the rates of increase of the outer diameter are different from each other.

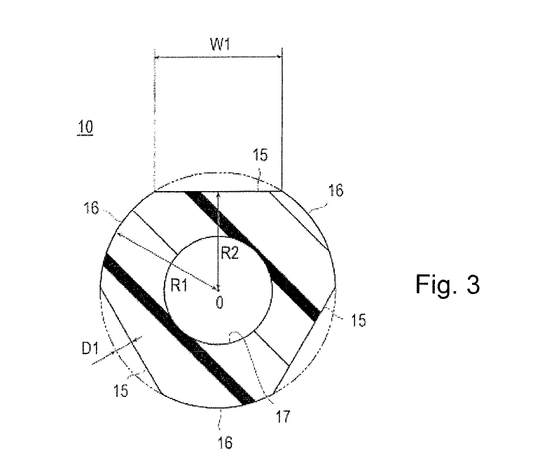

The distal portion 13 includes a tapered-shaped outer peripheral surface. The shaft portion 14 includes an approximately cylindrical outer peripheral surface. The largest outer diameter portion 16 is a boundary portion of the distal portion 13 and the shaft portion 14. The reduced cross-sectional area portion 15 has a cross-sectional area which is linear in the axial direction. In other words, the reduced cross-sectional area portion 15, when viewed in a cross-section which is parallel to the center axis of the dilator tube 11, defines a straight line. The reduced cross-sectional area portion 15 has a cross-sectional area which is linear in a circumferential direction as shown in FIG. 3. In other words, the reduced cross-sectional area portion 15, when viewed in a cross-section which is perpendicular to the center axis of the dilator tube 11, defines a straight line. The reduced cross-sectional area portion 15 is a thin and long elliptical flat surface. The reduced cross-sectional area portion 15 extends from the largest outer diameter portion 16 through part of the distal portion 13, and also extends from the largest outer diameter portion 16 through part of the shaft portion 14.

For example, the reduced cross-sectional area portion 15 is formed by chamfering of a part of an edge formed by intersection of the tapered-shaped outer peripheral surface of the distal portion 13 and the approximately cylindrical shaped outer peripheral surface of the shaft portion 14 from the proximal end of the distal portion 13 to the distal end of the shaft portion 14.

A length L1 of the distal portion 13 in the axial direction is, for example, 20 mm to 25 mm. A length L2 from the distal endmost portion of the dilator tube 11 to the distal end of the reduced cross-sectional area portion 15 in the axial direction is, for example, 8 mm to 15 mm. In addition, a length L3 of the reduced cross-sectional area portion 15 in the axial direction is, for example, 5 mm to 25 mm, and more preferably 8 mm to 15 mm.

As shown in FIG. 1, the reduced cross-sectional area portion 15 is equally divided between the distal portion 13 and the shaft portion 14, with the largest outer diameter portion 16 as the center.

As shown in FIG. 3, the dilator tube 11 includes three reduced cross-sectional area portions 15. The three reduced cross-sectional area portions 15 are disposed around a shaft center O at regular intervals. A width W1 of the reduced cross-sectional area portion 15 in a chord direction is equal to or greater than a depth D1 of the reduced cross-sectional area portion 15 in a direction perpendicular to the chord direction. The depth D1 is a difference between a length R1 from the shaft center O to a circular arc outer periphery which is different from the reduced cross-sectional area portion 15, and a length R2 of a perpendicular line from the shaft center O to the reduced cross-sectional area portion 15 (D1=R1−R2).

The width W1 is, for example, 0.2 mm to 5.0 mm, and more preferably 0.3 mm to 1.0 mm. The depth D1 is, for example, 0.01 mm to 1.0 mm, and more preferably 0.05 mm to 0.3 mm.

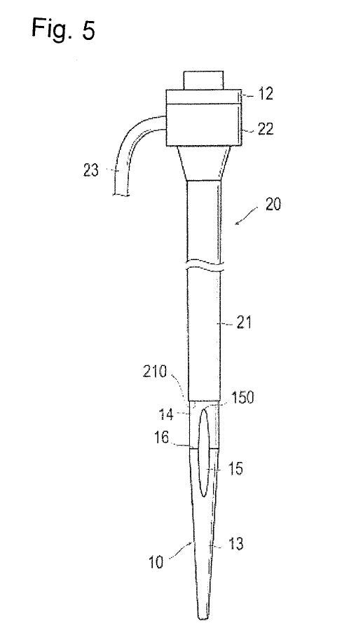

As shown in FIG. 5, a proximal portion 150 of the reduced cross-sectional area portion 15 is positioned at the distal end side with respect to a sheath distal end 210, when the dilator hub 12 of the dilator 10 is fixed with a sheath hub 22 of an introducer sheath 20.

Next, a diametrical expansion method of a hole performed by the dilator 10 will be described using insertion of a catheter into a blood vessel by the Seldinger method as an example.

First, an operator punctures a leg or an arm of a patient with a tubular needle (not shown) including a needle tip, to form a hole in the skin which communicates the outside with the blood vessel. Then, the operator inserts a guide wire into the blood vessel through the needle, while maintaining the insertion of the needle into the blood vessel. After the insertion of the guide wire, the operator extracts the needle. After that, the operator inserts the dilator 10 into the hole along the guide wire, to expand the diameter of the hole.

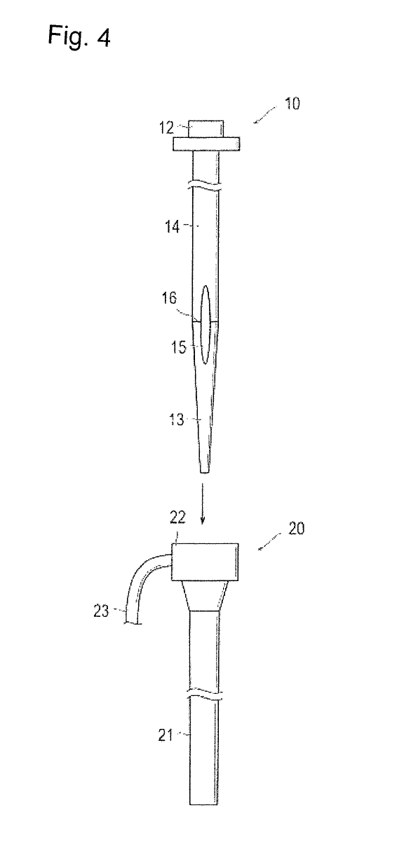

As shown in FIG. 4 and FIG. 5, the operator inserts the introducer sheath 20 and the dilator 10 into the hole, in a state where the dilator 10 is inserted into the tubular introducer sheath 20. At that time, the operator inserts the introducer sheath 20 and the dilator 10 into the hole along the guide wire, by passage of the guide wire through the inner cavity 17.

The introducer sheath 20 includes a flexible sheath tube 21, a sheath hub 22 which is fixed to a proximal end of the sheath tube 21, and a liquid injection tube 23 for liquid injection of saline or the like which communicates with the sheath tube 21 through the sheath hub 22.

The operator inserts the dilator 10 and the introducer sheath 20 into the hole, in a state where the entire reduced cross-sectional area portion 15 protrudes from the distal end of the sheath tube 21 and the dilator 10 and the introducer sheath 20 are fixed by the dilator hub 12 and the sheath hub 22. Since the dilator hub 12 is fixed to the sheath hub 22, the extraction of the dilator 10 from the distal end of the sheath tube 21 is prevented.

The dilator 10 passes through the hole and expands the diameter of the hole by the distal portion 13. Before the reduced cross-sectional area portion 15 reaches the hole, the entire outer periphery of distal portion 13 contacts the hole to expand the diameter of the hole. On the other hand, when the reduced cross-sectional area portion 15 reaches the hole, since the reduced cross-sectional area portion 15 does not easily contact the hole, the distal portion 13 mainly contacts the hole in the circular arc outer periphery portion other than the reduced cross-sectional area portion 15 to expand the diameter of the hole.

After the reduced cross-sectional area portion 15 passes through the hole, the distal end of the sheath tube 21 sequentially passes through the hole to be inserted into the blood vessel. After inserting the sheath tube 21 into the blood vessel by a desirable length, the operator extracts the dilator 10 while placing the sheath tube 21 in the blood vessel. The introducer sheath 20 is placed in the living body in a state where the proximal end is disposed to the outside of the body to exhibit a function of communicating the inside and the outside of the living body. The operator introduces a treatment tool such as a guide wire and a catheter into the living body through the introducer sheath 20 placed as described above.

According to the dilator 10, rapid change in resistance generated when the largest outer diameter portion 16 passes through the hole is suppressed by the reduced cross-sectional area portion 15, and as a result resistance sensed by an operator is reduced, and therefore smooth insertion of the dilator 10 can be performed.

The inventors practically confirmed such an effect in experiments performed using the dilator created based on the embodiment. The experiments will be simply described hereinafter.

The inventors created a dilator having a configuration the same as the embodiment, and a dilator which did not include a reduced cross-sectional area portion, that is, which included a circular arc edge over the entire periphery of the largest outer diameter portion, as a comparison target. In both dilators, the main configurations other than the reduced cross-sectional area portion were the same as each other. The width W1 of the reduced cross-sectional area portion was 1.050 mm, and the depth D1 of the reduced cross-sectional area portion was 0.163 mm (average value of depth D1 of three reduced cross-sectional area portions). The inventors obliquely inserted the dilators into a hole formed on a film obtained by imitating the skin, and measured changes in force applied to the dilators at that time.

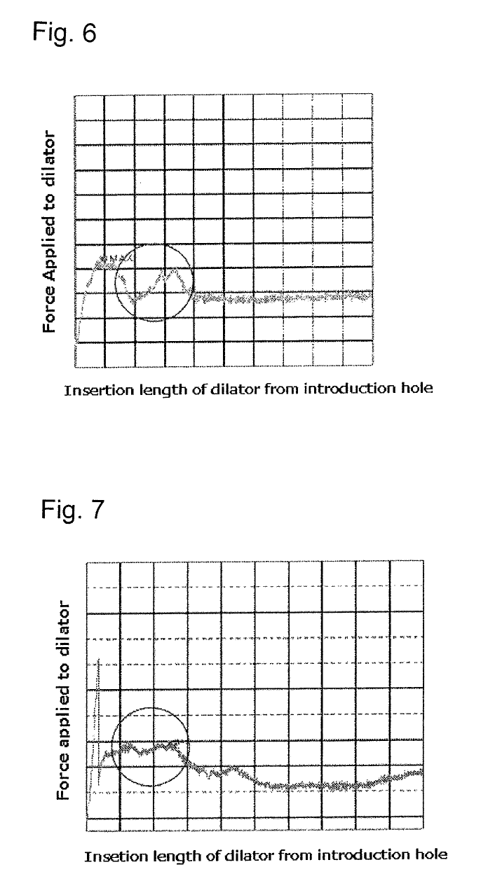

As shown in FIG. 6, in the experiment using the dilator not including the reduced cross-sectional area portion, rapid increase in resistance was confirmed, when the largest outer diameter portion passes through the hole (see portion surrounded by a circle). On the other hand, as shown in FIG. 7, in the experiment using the dilator including the reduced cross-sectional area portion, rapid increase in resistance described above was not confirmed (see portion surrounded by a circle). From this result, it was found that the rapid change in resistance generated when the largest outer diameter portion passes through the hole was suppressed by the reduced cross-sectional area portion. In addition, in the case of using the dilator including the reduced cross-sectional area portion, resistance when the largest outer diameter portion passes through the hole was also smaller than in the case without the reduced cross-sectional area portion. In the experimental result shown in FIG. 7, a peak appearing immediately after the starting of the insertion is caused by a burr remaining on the distal end of the dilator, and is not caused by the reduced cross-sectional area portion.

For comparison with the embodiment, the inventors created two kinds of dilators 30 and 40 based on the conventional technology disclosed above, and practically confirmed the effects thereof by the experiment.

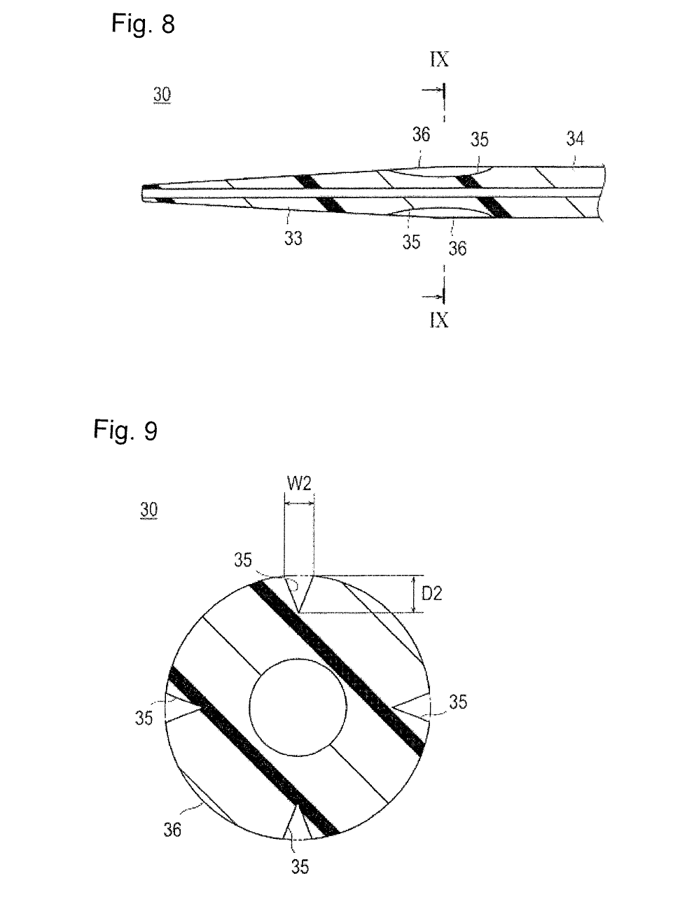

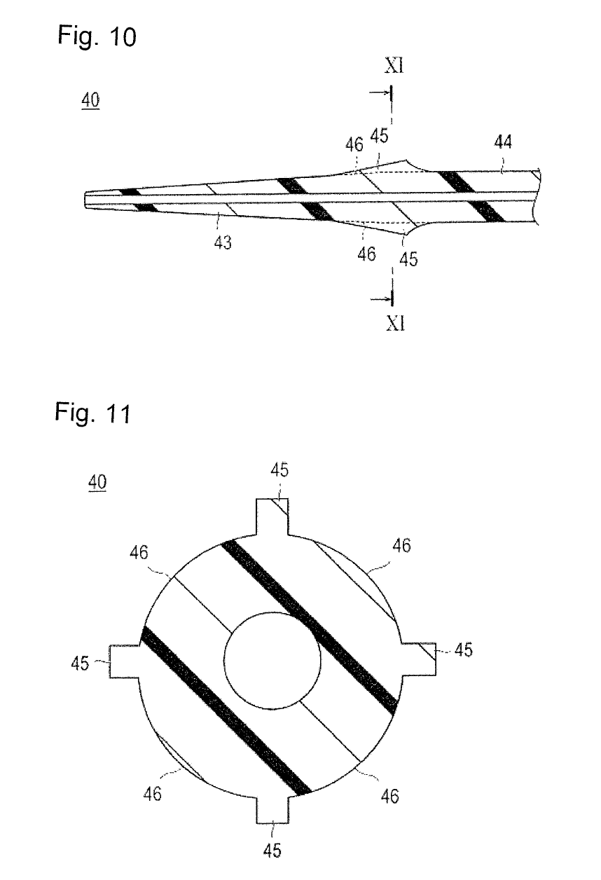

As shown in FIG. 8 and FIG. 9, the dilator 30 includes a slit-shaped groove 35 which passes through a largest outer diameter portion 36 and extends from a distal portion 33 to a shaft portion 34. A width W2 of the groove 35 is smaller than a depth D2 of the groove 35 (W2<D2). The width W2 of the groove 35 is 0.262 mm, and the depth D2 of the groove 35 is 0.315 mm (average value of depth D2 of four grooves 35). As shown in FIG. 10 and FIG. 11, the dilator 40 includes a protrusion 45 which passes through a largest outer diameter portion 46 and extends from a distal portion 43 to a shaft portion 44. The inventors obliquely inserted the dilators 30 and 40 into the hole formed on a film obtained by imitating the skin, and measured changes in force applied to the dilators 30 and 40 at that time.

As shown in FIG. 12, in a case of using the dilator 30, there is a sharp change in the graph before and after the largest outer diameter portion 36 passes through the hole, compared to the case of the embodiment shown in FIG. 6 (see portion surrounded by a circle). That is, the resistance changes largely. In addition, the resistance sensed when the largest outer diameter portion 36 passes through the hole is also large.

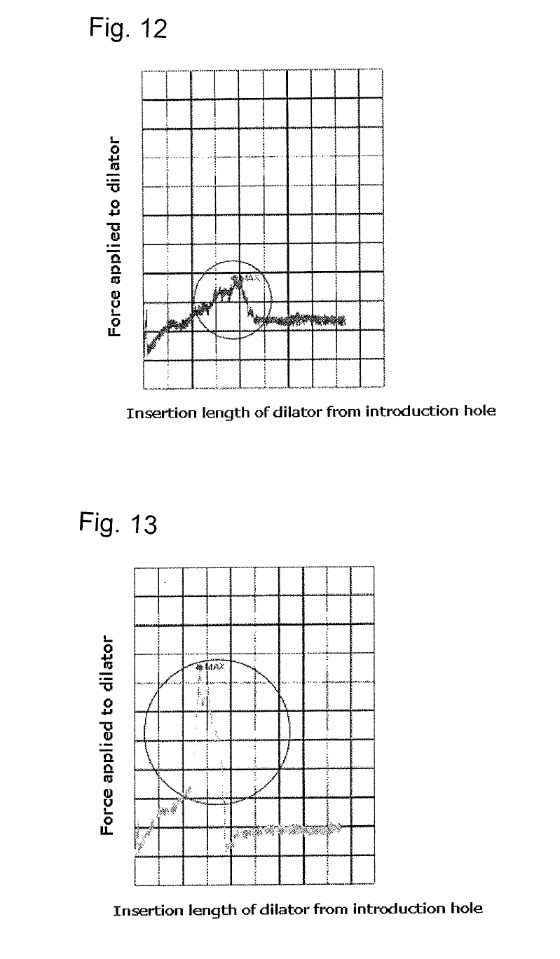

As shown in FIG. 13, in a case of using the dilator 40, there is a sharp change in the graph before and after the largest outer diameter portion 46 passes through the hole, compared to the case of the embodiment shown in FIG. 6 (see portion surrounded by a circle). That is, the resistance changes largely. In addition, the resistance sensed when the largest outer diameter portion 46 passes through the hole is also large.

From this result, it was confirmed that, with the slit-shaped groove 35 or the protrusion 45 having greater depth than the width as in the related art, the rapid change in resistance generated when the largest outer diameter portion passes through the hole cannot be suppressed unlike the embodiment, and the resistance sensed by the operator is also greater compared to the case of the embodiment.

The dilator 10 of the embodiment includes the plurality of reduced cross-sectional area portions 15, and accordingly, compared to the case of including one reduced cross-sectional area portion 15, the rapid change in resistance generated when the largest outer diameter portion 16 passes through the hole is more efficiently suppressed, and as a result the resistance sensed by the operator is further reduced, and therefore smoother insertion of the dilator 10 can be performed.

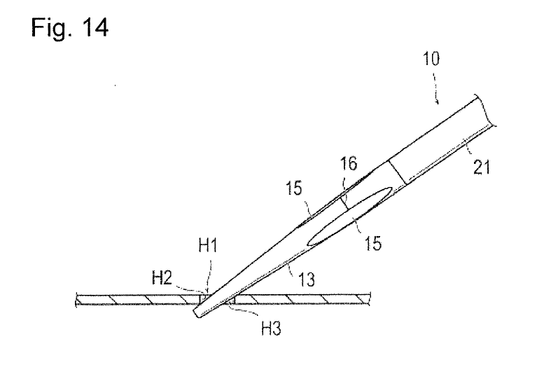

In addition, the reduced cross-sectional area portion 15 is provided in one of positions facing in the radial direction of the cross section of the largest outer diameter portion 16. In other words, in a cross-section passing through the largest outer diameter portion 16 and perpendicular to the center axis of the dilator tube 11, each reduced cross-sectional area portion 15 is radially opposite a part of the largest outer diameter portion 16, as illustrated in FIG. 3. Accordingly, when the operator inserts the dilator 10 obliquely with respect to a hole H1 as shown in FIG. 14, although a portion where the reduced cross-sectional area portion 15 in the largest outer diameter portion 16 is not provided (portion opposing the reduced cross-sectional area portion 15 in the radial direction, to describe with FIG. 3), passes through one of portions H3 and H2 in which rapid resistance change easily occurs and angles formed by the dilator 10 and the hole H1 opposing each other in the radial direction are an acute angle and an obtuse angle, the reduced cross-sectional area portion 15 can pass through the other portion thereof. Accordingly, the rapid change in resistance sensed when the largest outer diameter portion 16 passes through the hole H1 is easily and reliably suppressed, and therefore the function of the dilator 10 of reducing the resistance sensed by the operator at the time of insertion is readily realized.

Since the reduced cross-sectional area portion 15 has a cross-sectional area which is linear in the axial direction, change in angles between the hole and the distal portion 13 or the shaft portion 14 of the dilator 10 is moderated when the distal portion or the proximal portion of the reduced cross-sectional area portion 15 passes through the hole, and accordingly the resistance sensed by the operator can be reduced.

Since the reduced cross-sectional area portion 15 has a cross-sectional area which is linear in the circumferential direction, change in angles between the hole and the distal portion 13 or the shaft portion 14 is moderated in a case where the operator inserts the dilator 10 while twisting it, when the reduced cross-sectional area portion 15 passes through the hole, and accordingly the resistance sensed by the operator can be reduced.

Since the reduced cross-sectional area portion 15 is a thin and long elliptical flat surface, change in resistance in the axial direction or the circumferential direction generated when the distal portion 13 of the dilator 10 passes through the hole is suppressed.

As shown in the drawing, since the reduced cross-sectional area portion 15 is equally divided between the distal portion 13 and the shaft portion 14, with the largest outer diameter portion 16 as the center, change in resistance generated when the distal portion and the proximal portion of the reduced cross-sectional area portion 15 which is provided on the distal portion 13 of the dilator 10 pass through the hole, respectively, can be moderated.

### Second Embodiment

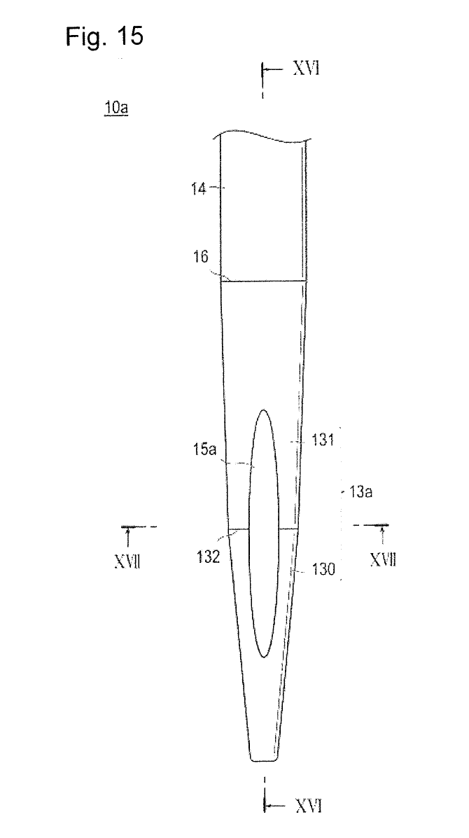

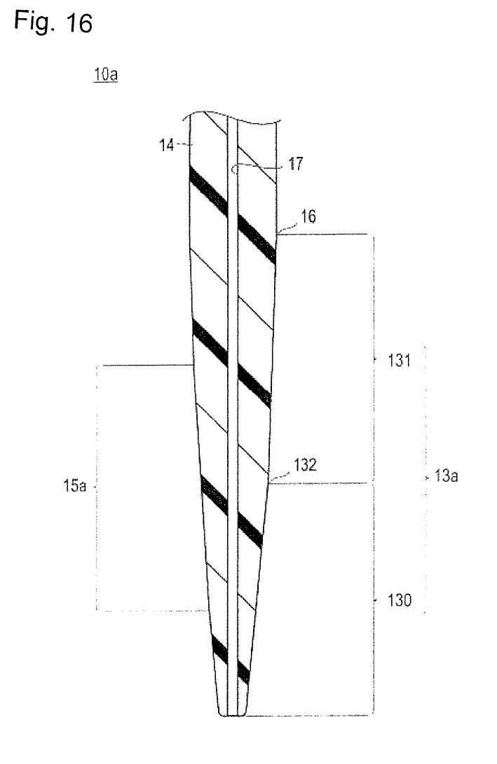

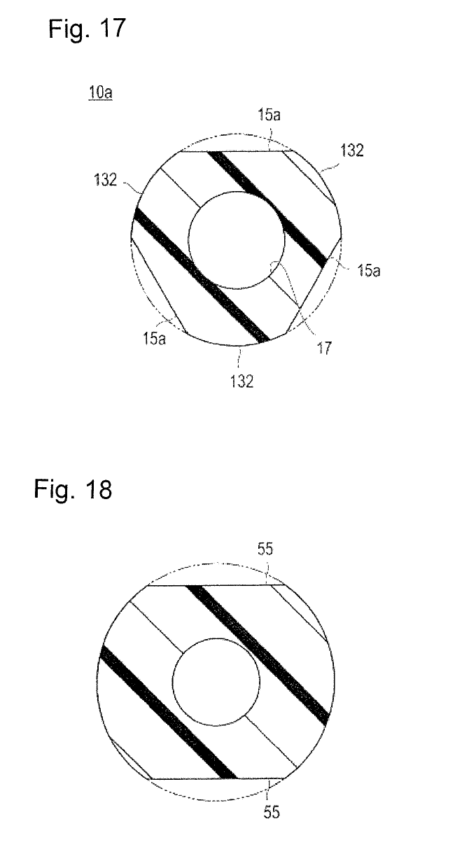

As shown in FIGS. 15 to 17, in a dilator 10a of a second embodiment, a shape of a distal portion 13a and a position of a reduced cross-sectional area portion 15a are different from those of the first embodiment. Since the other configurations and the method of use of the second embodiment are substantially the same as those of the first embodiment, overlapping descriptions will be omitted.

While the rate of increase of the outer diameter is constant in the distal portion 13 of the first embodiment, a rate of increase of an outer diameter changes in the distal portion 13a of the second embodiment. The distal portion 13a includes a first section 130 and a second section 131 which have a different rate of increase of an outer diameter from each other. The rate of increase of the outer diameter of the first section 130 provided on a distal end side is larger than the rate of increase of the outer diameter of the second section 131 provided on a proximal end side.

The reduced cross-sectional area portion 15a is provided on a boundary portion 132 of the first section 130 and the second section 131, that is, a portion in which the rate of increase of the outer diameter changes in the distal portion 13a, not on the largest outer diameter portion 16. In addition, the reduced cross-sectional area portion 15a extends to a distal end side and a proximal end side of the boundary portion 132. The reduced cross-sectional area portion 15a is provided on a position different from the reduced cross-sectional area portion 15 of the first embodiment, however the configuration of the reduced cross-sectional area portion 15a itself is substantially the same as the reduced cross-sectional area portion 15.

According to the dilator 10a, rapid change generated when the boundary portion 132 passes through the hole is suppressed by the reduced cross-sectional area portion 15a, and as a result resistance sensed by the operator is reduced, and therefore smooth insertion of the dilator 10a can be performed.

The dilator 10a includes the plurality of reduced cross-sectional area portions 15a, and accordingly, compared to the case of including one reduced cross-sectional area portion 15a, the rapid change in resistance generated when the boundary portion 132 passes through the hole is more efficiently suppressed, and as a result the resistance sensed by the operator is further reduced, and therefore smoother insertion of the dilator 10a can be performed.

In addition, the reduced cross-sectional area portion 15a is provided in one of positions facing in the radial direction of the cross section of the boundary portion 132. Accordingly, even when the portion in which the reduced cross-sectional area portion 15a is not provided passes through any one of the portions H2 and H3 of the hole in which the rapid change in resistance easily occurs, the reduced cross-sectional area portion 15a can pass through the other portion thereof. Accordingly, the rapid change in resistance generated when the boundary portion 132 passes through the hole is easily and reliably suppressed, and therefore the function of the dilator 10a of reducing the resistance sensed by the operator at the time of insertion is readily realized.

Since the reduced cross-sectional area portion 15a has a cross-sectional area which is linear in the axial direction, change in angles between the hole and the first section 130 or the second section 131 of the dilator 10a is moderated when the distal portion or the proximal portion of the reduced cross-sectional area portion 15a passes through the hole, and accordingly the resistance sensed by the operator can be reduced.

Since the reduced cross-sectional area portion 15a has a cross-sectional area which is linear in the circumferential direction, change in angles between the hole and the outer periphery of the first section 130 or the second section 131 is moderated in a case where the operator inserts the dilator 10 while twisting it, when the reduced cross-sectional area portion 15a passes through the hole, and accordingly the resistance sensed by the operator can be reduced.

Since the reduced cross-sectional area portion 15a is a thin and long elliptical flat surface, change in resistance in the axial direction or the circumferential direction generated when the distal portion 13a of the dilator 10a passes through the hole is suppressed.

As shown in the drawing, since the reduced cross-sectional area portion 15a is equally divided between the first section 130 and the second section 131, with the boundary portion 132 as the center, change in resistance generated when the distal portion and the proximal portion of the reduced cross-sectional area portion 15a which is provided on the distal portion 13a of the dilator 10a pass through the hole, respectively, can be moderated.

The present invention is not limited to the embodiments described above, and various modifications can be performed within a scope of the claims.

For example, the number of the reduced cross-sectional area portions is not limited to three, and may be one or four or more. The number of the reduced cross-sectional area portions can be suitably set based on viewpoints of improvement of intensity of the dilator and suppression of the change in resistance at the time of inserting into the hole. The plurality of reduced cross-sectional area portions may not be disposed symmetrically around the shaft center.

As shown in FIG. 18, the reduced cross-sectional area portions may be provided in both positions facing in the radial direction in the cross section of the largest outer diameter portion or in the cross section of the portion in which the rate of increase of the outer diameter changes in the distal portion. By providing the reduced cross-sectional area portions as described above, the reduced cross-sectional area portions can pass through both the portions H2 and H3 of the hole described above in which the rapid change in resistance easily occurs. Accordingly, the rapid change in resistance generated when the largest outer diameter portion passes through the hole is more efficiently suppressed.

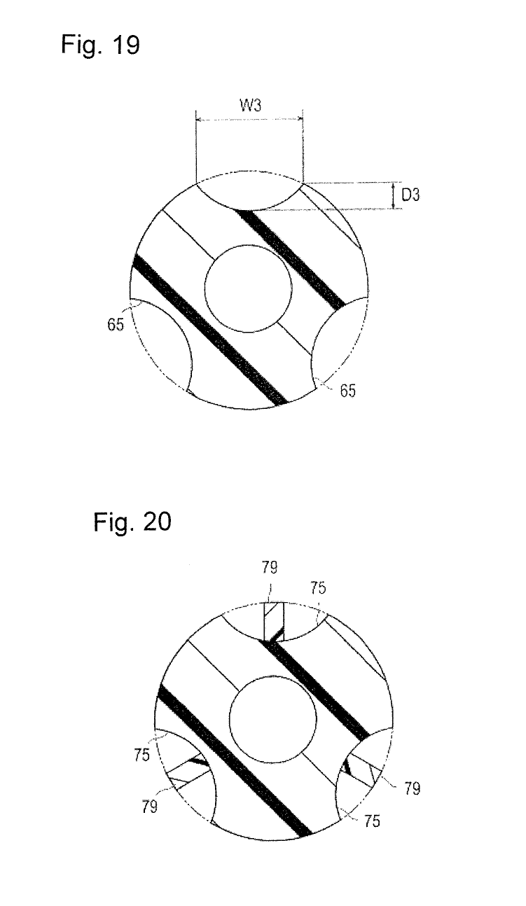

In addition, the reduced cross-sectional area portion is not limited to the flat surface of the embodiment, and may be a recessed curved surface 65, as shown in FIG. 19. In this case, a depth D3 of the reduced cross-sectional area portion is a distance from a chord connecting the cut circular arc (dashed-two dotted line in the drawing) to the deepest portion of the curved surface 65. In addition, the recessed shape is not limited to the circular arc as shown in the drawing, and may be a rectangular shape.

As shown in FIG. 20, the dilator may include reinforcing bodies 79 which are provided in reduced cross-sectional area portions 75. The reinforcing body 79 is, for example, a protrusion which extends in the axial direction of the dilator. The rapid change in resistance when increasing the intensity of the dilator and inserting the dilator into the hole can be suppressed by the reinforcing body 79.

The detailed description above describes a dilator. The dilator is disclosed by way of example. The invention is not limited, however, to the precise embodiment and variations described. Various changes, modifications and equivalents can effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A dilator comprising:
a distal portion, an outer diameter of which increases towards a proximal end side in an axial direction; and
a reduced cross-sectional area portion which is provided at least on a largest outer diameter portion of the distal portion, and is obtained by reducing a part of an outer periphery of a circular cross section,
wherein a width of the reduced cross-sectional area portion in a chord direction is equal to or greater than a depth of the reduced cross-sectional area portion in a direction perpendicular to the chord direction.

2. The dilator according to claim 1, wherein a plurality of the reduced cross-sectional area portions are provided.

3. The dilator according to claim 1, wherein the reduced cross-sectional area portion is provided in at least one of positions facing in a radial direction of the cross section of the largest outer diameter portion.

4. The dilator according to claim 2, wherein the reduced cross-sectional area portion is provided in at least one of positions facing in a radial direction of the cross section of the largest outer diameter portion.

5. A dilator comprising:
a distal portion, an outer diameter of which increases towards a proximal end side in an axial direction; and
a reduced cross-sectional area portion which is provided at least on a portion in which a rate of increase of the outer diameter of the distal portion changes, and is obtained by reducing a part of an outer periphery of a circular cross section,
wherein a width of the reduced cross-sectional area portion in a chord direction is equal to or greater than a depth of the reduced cross-sectional area portion in a direction perpendicular to the chord direction.

6. The dilator according to claim 5, wherein, in a cross-section passing through a largest outer diameter portion of the portion in which the rate of increase of the outer diameter of the distal portion changes and perpendicular to a center axis of the dilator tube, the reduced cross-sectional area portion is radially opposite a part of the largest outer diameter portion.

7. A dilator comprising:
a distal portion, an outer diameter of which increases towards a proximal end side in an axial direction; and
a reduced cross-sectional area portion which is provided on a portion in which a rate of increase of the outer diameter of the distal portion changes and on a largest outer diameter portion of the distal portion, and is obtained by reducing a part of an outer periphery of a circular cross section,
wherein a width of the reduced cross-sectional area portion in a chord direction is equal to or greater than a depth of the reduced cross-sectional area portion in a direction perpendicular to the chord direction.

8. The dilator according to claim 7, wherein, in a cross-section passing through a largest outer diameter portion of the portion in which the rate of increase of the outer diameter of the distal portion changes and perpendicular to a center axis of the dilator tube, the reduced cross-sectional area portion is radially opposite a part of the largest outer diameter portion.
